(19) United States
(12) Patent Application Publication
(10) Pub. No.: US 2005/0277921 A1
(43) Pub. Date: Dec. 15, 2005

(54) Prosthetic joint and nucleus supplement

(75) Inventors: Lukas Eisermann, Memphis, TN (US); Eddie Ray, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 10/856,730

(22) Filed: May 28, 2004

(51) Int. Cl. A61B 17/58; A61F 2/44; A61F 2/46; A61B 17/86

(52) U.S. Cl. 606/61; 623/17.11; 623/17.16; 606/73; 623/23.44; 623/908; 606/60

(57) ABSTRACT

The present disclosure provides improved prosthetic joints and a nucleus supplement device for a spinal disc without violating its annulus fibrosus. In one example, a prosthetic joint includes: a tip adapted for mating against a superior articular process of an inferior vertebra, and the tip acts as at least a spacer between the superior articular process and an inferior articular process of a superior vertebra; and an body for mating against a hole in the superior vertebra, and the body comprises a surface that is uneven along its entire length. In another example, a method for restoring motions of a joint includes: providing a body for a prosthetic joint; threading a surface of the body, and the threading extends an entire length of the body; and creating an internal cavity in the prosthetic joint.

### FIELD OF THE INVENTION

The present disclosure relates generally to artificial replacement devices and supplements, and more particularly, to prosthetic joints and nucleus supplementation.

### BACKGROUND

Facet joints are also called zygapophyseal joints. They are located in the posterior column of the spine and on the tips of the articular processes. They are formed by the articular processes of adjacent vertebrae—the inferior articular process of a vertebra articulates with the superior articular process of the vertebra below. Facet joints are synovial gliding joints because the articular surfaces glide over each other. They are important in stabilizing the spine, and carry approximately 20% of the compressive load on the spine. Accordingly, their anatomic position and orientation affect the mobility of each spinal region. For example, in the cervical region, facet joints are oriented in the coronal plane and are capable of a significant range of motion in the six degrees of freedom. In the lumbar area, the facet joints are oriented in the sagittal plane.

Major trauma, repetitive minor trauma, or many other factors may cause a facet joint to degenerate. As a result, the hyaline cartilage that lines the joint will lose its water content, and eventually becomes worn out completely. Then, the articular processes begin to override each other as the joint capsules become stretched, resulting in the malalignment of the joints and abnormal biomechanical function of the motion segment.

Since facet joints work with discs to support spinal loads, an injured or traumatized disc may also cause the joints to degenerate. As a person ages, discs often experience anatomical changes. By the age of fifty, over 95% of the people will exhibit evidence of disc degeneration. The most significant alterations to the disc include the decrement of water and proteoglycan content of its nucleus pulposus. As a result, the disc begins to lose its normal height, and becomes less resistant and resilient to loading forces. In particular, the nucleus pulposus looses the ability to sustain hydrostatic pressure. In essence, the disc no longer fully acts like a shock absorber between the vertebral bodies. To cope with the degraded disc, load is transferred from the central nucleus to the peripheral annulus, resulting in loading changes to the vertebral facets and damages to joints. For example, a decreased disc height results in overriding of the facets, causing loss of cartilage and a hypertrophic process on the articular surfaces. Given time, the natural adaptive processes may significantly re-model the facet joint anatomy.

Previous treatments of degenerated joints possess many problems. For example, in many instances, treatments emphasize the anterior, but not the posterior column of the spine. Also, spinal fusion has been widely used to repair damaged discs. However, fusion decreases joint functions by limiting the range of motions in flexion, extension, rotation, and lateral bending at the affected level. At the levels adjacent to a fused level, the disc is exposed to abnormal stresses and hypermobility.

Previous treatments of degenerated nucleus pulposus also possess a number of problems. For example, nucleus replacements have been utilized to treat degenerated nucleus pulposus. However, those replacements cause damages to discs by violating the annulus fibrosus.

### SUMMARY

In one embodiment, a prosthetic joint comprises: a tip adapted for mating against a superior articular process of an inferior vertebra wherein the tip acts as a spacer between the superior articular process and an inferior articular process of a superior vertebra; and an elongated body for mating against a hole in the superior vertebra wherein the body comprises a surface that is uneven along its length.

In another embodiment, a method for restoring motions of a joint comprise: providing a body for a prosthetic joint; threading a surface of the body wherein the threading extends an entire length of the body; and creating an internal cavity in the prosthetic joint.

In a third embodiment, a method for spacing intervertebral facets to prevent bone-on-bone grinding comprises: applying a material between a superior facet of an inferior vertebra and an inferior facet of a superior vertebra wherein the material provides articulation.

In a fourth embodiment, a method for pressurizing nucleus pulposus, comprise: providing a device for delivering a substance into a disc space wherein the substance pressurizes nucleus pulposus of a disc without violating annulus fibrosus of the disc.

### BRIEF DESCRIPTION OF THE DRAWINGS

FIG. 1 is a side view of a prosthetic joint according to one embodiment of the present disclosure.

FIG. 2A illustrates a prosthetic joint and vertebrae according to one embodiment of the present disclosure.

FIG. 2B illustrates a prosthetic joint replacement according to one embodiment of the present invention.

FIG. 3 is a section view of a prosthetic joint according to one embodiment of the present disclosure.

FIG. 4 illustrates generally an osmotic balloon used for a prosthetic device according to one embodiment of the present disclosure.

FIG. 5 illustrates a nucleus supplement device and vertebrae according to one embodiment of the present disclosure.

FIG. 6 is a section view of a prosthetic joint according to one embodiment of the present disclosure.

### DETAILED DESCRIPTION

For the purposes of promoting an understanding of the principles of the invention, references will now be made to the embodiments, or examples, illustrated in the drawings and specific languages will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The present disclosure provides improved prosthetic joints for an animal subject. The present disclosure further provides a nucleus supplement device for a spinal disc without violating its annulus fibrosus.

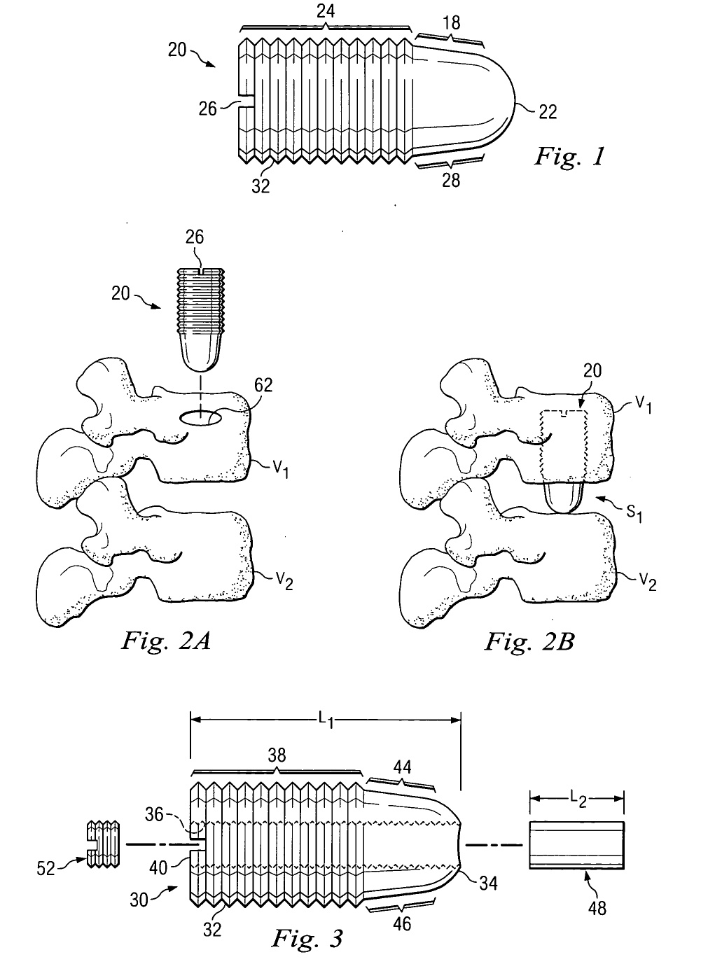

Referring now to FIG. 1, in one embodiment, a prosthetic joint 20 includes a tip 22, an elongated body 24, and an opening 26.

The tip 22 may be partially cylindrical, and is adapted for mating against a superior articular process of an inferior vertebra. It acts as a spacer between the superior articular process and an adjacent inferior articular process of a superior vertebra. By providing a spacer between the superior and inferior articular processes, bone grinding is eliminated, and natural spatial relationship is restored. In addition, under this posterior approach, burdens on a disc will be better shared by a posterior column of the vertebrae. As a result, the disc will become less overburdened, and its degeneration will be slowed.

Portions 18 and 28 of the tip 22 may be tapered or otherwise modified to articulate against cartilage of the vertebrae, so that during movements, the tip 22 will maintain its proper position. It is contemplated that the tip 22 may comprise a variety of shapes, such as a cylinder, sphere, partial sphere, partial cone, or partial pyramid. It is also contemplated that the tip 22 may comprise any suitable biocompatible material, such as, but without limitation, metal, plastics, ceramics, polymers, carbon fiber, shape memory alloys, composites, allograft or porous material.

The body 24 may be substantially cylindrical. Its surface 32 is uneven, and may be created by threading, roughening, or machining, so that the body 24 may engage a hole in the superior vertebra. In this illustration, the surface 32 is threaded, and the threads are substantially even. It is also contemplated that the threads may be uneven. It is further contemplated that the surface 32 may be modified in other ways to engage the superior vertebra.

The body 24 may comprise any suitable biocompatible material, such as, but without limitation, metal, plastics, ceramics, polymers, carbon fiber, shape memory alloys, composites, allograft or porous material. It may be adapted for osseo-integration to facilitate its bonding with the superior vertebra. For example, it may comprise a hydroxyapatite or collagen coating. In another example, it may comprise carbon fiber or biomimetic bone, or may be anodized.

The opening 26 may comprise any shape, such as a hexagon or cross, to allow any suitable tool or instrument (not shown), which may be a screw driver, to drive the prosthetic joint 20.

In one embodiment, the prosthetic joint 20 is created from a single object, which comprises any suitable biocompatible material, such as stainless steel, polymers, carbon fiber, shape memory alloys, or porous material. The opening 26 may be produced by cutting off unwanted portions of the single object. As a result, movement of the tip 22 relative to the body 24 may be constrained, and the wear and tear of the contact surface between the tip 22 and the body 24 may be limited. It is also contemplated that the tip 22 and the body 24 may be created from separate objects. It is contemplated that an allograft plug with a demineralized tip may be used to form the prosthetic joint 20.

Utilization of the present disclosure will now be briefly described. It will be understood that access to a facet joint space and vertebrae preparation are known in the art and will be described only briefly herein. It will also be understood that a medial/dorsal approach is known in the art, and will not be described in details herein. Referring now to FIG. 2A, in one embodiment, the prosthetic joint 20 is inserted from a medial/dorsal approach. In operation, The joint 20 may be loaded into a delivery tube or sleeve, and then placed adjacent to a hole 62 that is drilled inside a superior vertebra V1. A screw driver may be used in the opening 26 to forcibly urge the disc replacement device 20 into the hole 62, until the tip 22 mates against a superior articular process of an inferior vertebra V2. At that point, the joint 20 acts as a spacer between the superior articular process of the inferior vertebra V2 and an inferior articular process of the superior vertebra V1.

It is contemplated that other approaches, such as a lateral approach, bilateral approach, and optionally, with visualization approach may also be utilized to insert the prosthetic joint.

Insertion preparation may be tailored to the condition of a diseased joint. For example, all or a part of cartilage may be removed. Alternatively, cartilage may simply be left for mating against a prosthetic joint. In one embodiment, insertion preparation may comprise drilling the hole 62 in the superior vertebra V1, and removing materials from the vertebrae V1 and V2 for mating against the tip 22 of the prosthetic joint 20. The hole 62 may be threaded, roughened or machined in its surface to engage the surface 32 of the prosthetic joint 20. It is also contemplated that the tip 22 may be adapted to an existing superior articular process of the inferior vertebra V2, so that preparation of the inferior vertebra V2 may be limited.

In one embodiment, a portion of the superior articular process of the inferior vertebra V2 has been prepared to create a partial cylindrical area to receive the partially cylindrical tip 22, so that the tip 22 will substantially abut the prepared inferior vertebra V2.

In another embodiment, the prepared portion of the superior articular process of the inferior vertebra V2 may be limited to the area necessary to receive the prosthetic joint 20, and the rest of the superior articular process remains unprepared. The unprepared portions of the superior articular process may engage the tip 22 to resist expulsion of the prosthetic joint 20 from its proper position.

Referring now to FIG. 2B, shown therein is the prosthetic joint 20 placed within an animal body according to one embodiment of the present disclosure. In this embodiment, a distance S1 between the vertebrae V1 and V2 may be decreased by the placement of the prosthetic joint 20, and thus reduce the pain the animal body may experience in that area. However, it is also contemplated that the distance S1 may not be reduced due to the insertion of the prosthetic joint 20.

Referring now to FIG. 3, in one embodiment, some components of the prosthetic joint 20 may be modified to create a prosthetic joint 30, which may include a tip 34, a body 38, a cavity 36, and an opening 40.

The tip 34 is otherwise partially cylindrical, but interrupted by the cavity 36. It includes tapered portions 44 and 46, which may be adapted for mating against cartilage of vertebrae. The tapered portions 44 and 46 may also ease the insertion of the prosthetic joint 30 into an animal body.

The cavity 36 may be used for delivering a substance, which may comprise any suitable biocompatible substance, such as hydrogel, silicone, polyurethane, collagen, or bone morphogenic protein into an articular capsule and/or joint space. The cavity 36 may reside inside the body 38, and has a length L1 that may extend the entire combined lengths of the body 38 and the tip 34. Prior to implanting the prosthetic joint 30, the cavity 36 may be loaded with a rod 48, which may extend a length L2. The length L2 may be smaller than or equal to the length L1. It is contemplated that the rod 48 may comprise any suitable biocompatible materials, such as hydrogel, silicone, polyurethane, collagen, allograft cartilage, or other natural or synthetic materials. A conventional driver device 52, which may be a set screw, may be used to advance the rod 48 to force hydrogel into an articular capsule and/or a joint space.

In another embodiment, the outer surface of the rod 48 may be roughened, machined, or threaded along its entire length L2. Likewise, a surface of the cavity 36 may be roughed, machined, or threaded to engage the rod 48.

Utilization of the prosthetic joint 30 will now be briefly described. In one embodiment, the prosthetic joint 30 may be loaded into a delivery tube or sleeve, and then placed adjacent to a hole in a superior vertebra. It may be inserted through the hole, and advanced until its tapered portions 44 and 46 mate against cartilage of vertebrae. At that point, a conventional tool, which may be a screw driver, may be used with the driver device 52 to advance the rod 48 toward the tip 34, forcing a portion of the rod 48 into an articular capsule. The rod 48 may be advanced until it contacts the articular capsule, or further than the contacting point, so that the rod 48 pushes against the articular capsule. After the hydrogel settles into the articular capsule, it will grow in size and pressurize the articular capsule. Thereafter, the screw driver and the driver device 52 may be removed from the animal body. Alternatively, they may be left inside the animal body. In that case, each of them may comprise a suitable biocompatible material, which may be stainless steel or carbon fiber.

The hydrogel in the articular capsule and/or joint space may function as a spacer between an inferior articular process of a superior vertebra and a superior articular process of an inferior vertebra. It may pressurize an articular capsule, and provide articulation. As a result, bone-on-bone grinding of the adjacent facets may be eliminated. After a certain period of time, which may be six months, one year, or based on clinical diagnosis, the hydrogel may creep into other areas, or deform severely. At that point, the driver device 52 may be advanced further toward the tip 34, forcing another portion of the rod 48 into the articular capsule to replace the deformed hydrogel. Such procedure may be repeated a plurality of times, if necessary, each time advancing an additional portion of the rod 48 into the articular capsule.

Insertion preparation may be made by drilling the hole in the superior vertebra and removing materials from the vertebrae for mating against the tip 34 of the prosthetic joint 30. Further, the hole may be threaded along its surface to engage the roughened surface of the prosthetic joint 30. It is contemplated that the tip 34 may be adapted to an existing superior articular process of the inferior vertebra, so that the preparation for the inferior vertebra may be limited.

In one embodiment, a portion of the superior articular process of the inferior vertebra has been prepared to create a partial cylindrical area to receive the tapered potions 44 and 46, so they may substantially abut the prepared inferior vertebra.

In another embodiment, the prepared portion of the superior articular process of the inferior vertebra may be limited to the area necessary to receive the prosthetic joint 30, and the rest of the superior articular process remains unprepared. The unprepared portions of the superior articular process may engage the tip 34 to resist expulsion of the prosthetic joint 30 from its proper position.

The rod 48 may be advanced into an articular capsule and/or joint space through many means. For example, it can be advanced via any conventional mechanical means, which may employ a biocompatible screw driver (not shown). The screw driver may be left inside an animal subject between treatments. Alternatively, it may be inserted each time to further advance the rod 48 into the articular capsule and/or joint space.

In another embodiment, non-invasive methods may be employed to advance the rod 48. In one example, an infusion pump is utilized. Upon receiving an external signal, which may be a radio frequency signal or an ultrasound excitation, the infusion pump will advance the rod 48 by conventional means, such as an electronic motor or a pressure system. It will be understood that the infusion pump is known in the art, and will not be described further herein.

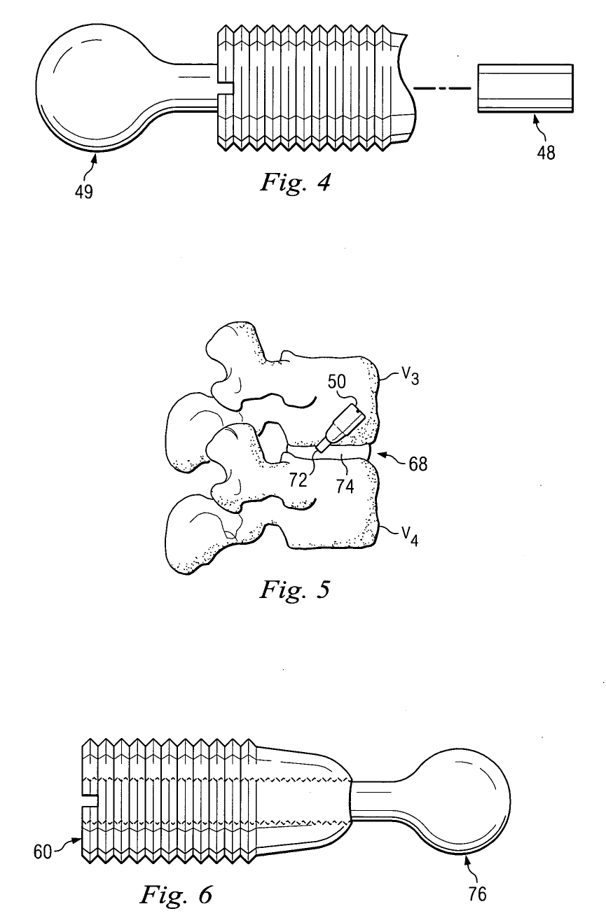

Referring generally to FIG. 4, in one embodiment, an osmotic balloon 49 may be utilized to drive the rod 48 into an articular capsule. The osmotic balloon may comprise a biocompatible membrane, which allows water, but not larger articles, to permeate through. The balloon 49 may be used as an electrolyte reservoir, and may contain osmotically active electrolyte, such as salt or salt water, and/or hyaluronic acid. As a result, pressure from the active electrolyte will drive the rod 48 into an articular capsule, so that a pressure equilibrium may be maintained between both sides of the balloon 49. Thereafter, the balloon may remain inside an animal body. For example, it may reside between back muscles of the animal body.

In another embodiment, an infusion pump, which is connected through a valve to an osmotic balloon, may be combined with the osmotic balloon to drive the rod 48 into an articular capsule. As the infusion pump receives a control signal, it may release additional osmotically active electrolyte into the balloon, resulting in an increased pressure. The increased pressure may drive the rod 48 further into an articular capsule, so that the pressures on both sides of the osmotic balloon may be equalized. The infusion pump may be made of any biocompatible material, and may be left inside an animal body between repeated advancements of the rod 48.

Referring now to FIG. 5, in one embodiment, to treat a degenerated disc, an anterior approach of pressurizing its nucleus pulposus may be adopted. In this embodiment, the prosthetic joint 30 may be modified to created an identical, but larger prosthetic device 50. In operation, an angled approach may be employed to insert the prosthetic device 50 into a disc space of an animal body. A hole may be drilled in a superior vertebra V3 (including its endplate) to accommodate the insertion of the prosthetic device 50, and its surface may be roughened to engage a roughened body surface of the prosthetic device 50. After a tip of the prosthetic device 50 penetrates the endplate and settles into a disc space 68, a rod 72, which may comprise any suitable biocompatible substance, such as hydrogel, silicone, polyurethane or collagen, may be advanced by a driver device towards a disc 74 to repressurize its nucleus pulposus without violating its annulus fibrosus. The rod 72 may be advanced until it contacts the disc 74, or may be advanced further to pressurize the disc 74. As a result, the nucleus pulposus will be revitalized, and its degeneration will be slowed. After a certain period, which may be six months, one year, or based on diagnosis or a patient's experiencing pain, the exposed portion of the rod 72 may become deformed and lose its pressurizing function. At that point, the rod 72 may be advanced further to repressurize the nucleus pulposus of the disc 74. And such advancement may be repeated several times, if necessary. Between treatments, the prosthetic device 50 may be left in the animal body, or reinserted into the animal body during each treatment. The driver device may be any conventional tool, which may be a biocompatible screw driver. It may be left inside the animal body along with the prosthetic device 50. Alternatively, the driver device may be removed after each treatment, and reinserted during a subsequent treatment.

To provide sufficient volume of hydrogel for the disc 74, it is contemplated that the prosthetic device 50 may be replicated, and a plurality of the prosthetic device 50 may be used to repressurize the nucleus pulposus of the disc 74.

Similar to the descriptions with respect to the rod 48, the rod 72 may be advanced into the disc space 68 through many methods. In one embodiment, non-invasive methods may be employed to advance the rod 72. For example, an infusion pump may be utilized. Upon receiving an external signal, such as a radio frequency signal or an ultrasound excitation, the infusion pump may advance the rod 72 by any conventional means, such as an electronic motor or a pressure system. In another example, a combination of an infusion pump and an osmotic balloon may be utilized to advance the rod 72.

Referring now to FIG. 6, in one embodiment, some components of the prosthetic joint 30 of FIG. 3 may be modified to create a prosthetic joint 60. In the illustrated example, a membrane 76, which may be a balloon, may replace the rod 48. The membrane 76 may comprise any biocompatible material, such as rubber, silicon rubber, shape memory alloys, titanium, carbon fiber, polymers, stainless steel, or porous material. It may be filled partially or completely with a soft, viscous material, such as silicon gel, hydrogel, polyurethane, or collagen. The membrane 76 may be advanced into an articular capsule by the methods described previously with respect to the rod 48.

Although only a few exemplary embodiments of this invention have been described in details above, those skilled in the art will readily appreciate that many modifications are possible in the exemplary embodiments without materially departing from the novel teachings and advantages of this invention. For example, prosthetic joints disclosed herein may be utilized in combination with disc replacement(s). Also, features illustrated and discussed above with respect to some embodiments can be combined with features illustrated and discussed above with respect to other embodiments. Accordingly, all such modifications are intended to be included within the scope of this invention.

1. A prosthetic joint for use between a first vertebra and a second vertebra having a bearing surface, comprising:
a body for mating against an opening in a first vertebra; and
a tip connected to the body adapted for mating against a bearing surface of a second vertebra wherein the tip acts as at least a partial spacer between the first and second vertebrae.

2. The prosthetic joint of claim 1 wherein the tip is adapted for mating against a first articular process of the second vertebra, wherein the tip acts as the spacer between the first articular process and an articular process of the first vertebra.

3. The prosthetic joint of claim 1 wherein the body comprises an uneven surface.

4. The prosthetic joint of claim 3 wherein the uneven surface is created by threading.

5. The prosthetic joint of claim 1 further comprising an opening opposing the tip wherein the opening is adapted for driving the prosthetic joint with an instrument.

6. A method for manufacturing a prosthetic joint for use between a first vertebra and a second vertebra having a bearing surface, the method comprising:
supplying a body for mating against an opening in a first vertebra; and
adapting a tip for mating against a bearing surface of a second vertebra wherein the tip acts as a spacer between the first and second vertebrae.

7. The method of claim 6 wherein the adapting comprises shaping the tip for mating against an articular process of the second vertebra.

8. The method of claim 6 further comprising creating an uneven surface for the body.

9. The method of claim 6 further comprising creating an opening opposing the tip wherein the opening is for driving the prosthetic joint with an instrument.

10. A prosthetic joint for use between a first vertebra and a second vertebra having a bearing surface, comprising:
a body for mating against an opening in a first vertebra;
a tip connected to the body wherein portions of the tip are adapted for mating against a bearing surface of a second vertebra; and
an internal cavity within the tip for delivering a substance to the second vertebra.

11. The prosthetic joint of claim 10 wherein the internal cavity extends from the body to the tip.

12. The prosthetic joint of claim 10 wherein the substance comprises a natural material.

13. The prosthetic joint of claim 10 wherein the substance comprises allograft cartilage.

14. The prosthetic joint of claim 10 wherein the substance comprises a synthetic material.

15. The prosthetic joint of claim 10 wherein the substance comprises hydrogel.

16. The prosthetic joint of claim 10 wherein the substance comprises silicone.

17. The prosthetic joint of claim 10 wherein the substance comprises polyurethane.

18. The prosthetic joint of claim 10 wherein the substance comprises collagen.

19. The prosthetic joint of claim 10 wherein the substance comprises a balloon, wherein the balloon is formed of an expansible material.

20. A prosthetic joint for use between a first vertebra and a second vertebra having a bearing surface, comprising:
a body for mating against an opening in a first vertebra; and
means for mating against a bearing surface of a second vertebra.

21. The prosthetic joint of claim 20 wherein the means for mating is disposed adjacent to a distal end of the prosthetic joint.

22. The prosthetic joint of claim 20 wherein the body comprises a first material, wherein the means for mating comprises a second material.

23. The prosthetic joint of claim 20 further including means for delivering a substance to the second vertebra.

24. The prosthetic joint of claim 23 wherein the substance comprises a natural material.

25. The prosthetic joint of claim 23 wherein the substance comprises a synthetic material.

26. A method for inserting a prosthetic joint for use between a first vertebra and a second vertebra having a bearing surface, comprising:
inserting a tip through an opening in a first vertebra until at least a portion of the tip mates against a bearing surface of a second vertebra.

27. The method of claim 26 further comprising inserting at least a portion of a body through the opening, so that at least a portion of the body mates against the opening.

28. The method of claim 26 further comprising delivering a substance through an opening within the tip to the second vertebra.

29. The method of claim 26 further comprising delivering a natural material through an opening within the tip to the second vertebra.

30. The method of claim 26 further comprising delivering a synthetic material through an opening within the tip to the second vertebra.

31. The method of claim 26 further comprising delivering hydrogel through an opening within the tip to the second vertebra.

32. The method of claim 26 further comprising delivering silicone through an opening within the tip to the second vertebra.

33. The method of claim 26 further comprising delivering polyurethane through an opening within the tip to the second vertebra.

34. The method of claim 26 further comprising delivering collagen through an opening within the tip to the second vertebra.

35. The method of claim 26 further comprising:
delivering at least a portion of a balloon to the second vertebra.

36. The method of claim 26 further comprising loading a substance in an internal cavity within the tip.

37. The method of claim 36 further comprising using a mechanical method to advance the substance to the second vertebra.

38. The method of claim 36 further comprising using a mechanical method to advance the substance into an articular capsule of the second vertebra.

39. The method of claim 36 further comprising using an osmotic balloon to advance the substance to the second vertebra.

40. The method of claim 36 further comprising using an osmotic balloon to advance the substance into an articular capsule of the second vertebra.

41. The method of claim 36 further comprising using an infusion pump to advance the substance to the second vertebra.

42. The method of claim 36 further comprising using an infusion pump to advance the substance into an articular capsule of the second vertebra.

43. The method of claim 36 further comprising using a combination of an osmotic balloon and an infusion pump to advance the substance to the second vertebra.

44. The method of claim 36 further comprising using a combination of an osmotic balloon and an infusion pump to advance the substance into an articular capsule of the second vertebra.

45. A method for spacing intervertebral facets to prevent bone-on-bone grinding, the method comprising:
delivering a material between a superior facet of an inferior vertebra and an inferior facet of a superior vertebra wherein the material provides articulation.

46. The method of 45 wherein the delivering includes flowing the material between the superior facet and the inferior facet.

47. The method of 45 furthering comprising creating a passage through the superior vertebra into a space between the superior facet and the inferior facet.

48. The method of 45 furthering comprising delivering a device into a substantially intact facet joint.

49. The method of 45 wherein the material comprises a natural substance.

50. The method of 45 wherein the material comprises a synthetic substance.

51. The method of 45 wherein the material comprises hydrogel.

52. The method of 45 wherein the material comprises silicone.

53. The method of 45 wherein the material comprises polyurethane.

54. The method of 45 wherein the material comprises collagen.

55. The method of 28 wherein the applying comprises providing a balloon wherein the balloon is filled at least partially with an expansible material.

56. A method for pressurizing nucleus pulposus, the method comprising:
delivering a substance into a disc space wherein the substance pressurizes nucleus pulposus of a disc without violating annulus fibrosus of the disc.

57. The method of claim 56 wherein the substance comprises a natural substance.

58. The method of claim 56 wherein the substance comprises a synthetic substance.

59. The method of claim 56 wherein the substance comprises hydrogel.

60. The method of claim 56 wherein the substance comprises silicone.

61. The method of claim 56 wherein the substance comprises silicone.

62. The method of claim 56 wherein the substance comprises collagen.
